(19) United States
(12) Patent Application Publication
(10) Pub. No.: US 2020/0158671 A1
(43) Pub. Date: May 21, 2020

(54) GAS SENSOR

(71) Applicant: LG ELECTRONICS INC., Seoul (KR)

(72) Inventors: Insung HWANG, Seoul (KR); Moosub KIM, Seoul (KR); Sungeun LEE, Seoul (KR); Sunghee LEE, Seoul (KR)

(73) Assignee: LG ELECTRONICS INC., Seoul (KR)

(21) Appl. No.: 16/635,318

(22) PCT Filed: Mar. 22, 2018
(86) PCT No.: PCT/KR2018/003339

§ 371 Date: Jan. 30, 2020

Related U.S. Application Data
(60) Provisional application No. 62/538,781, filed on Jul. 30, 2017.

(30) Foreign Application Priority Data
Dec. 6, 2017 (KR) 10-2017-0166905

(51) Int. Cl. G01N 27/12; G01N 33/00

(52) U.S. Cl. CPC G01N 27/12; G01N 33/0047

(57) ABSTRACT

The present disclosure relates to a gas sensor that minimizes the effects of humidity. The present disclosure relates to a gas sensor that minimizes the effects of humidity. Provided, in the present disclosure, is a gas sensor comprising: a substrate; a unit sensing unit disposed on the substrate and including a wiring electrode, a heater electrode, and a gas sensing material; a protective cap having a side wall part formed on the substrate and surrounding the unit sensing unit and a cover part disposed above the unit sensing unit and including at least one hole; and a heater unit disposed between the unit sensing unit and the cover unit, wherein the heater unit generates heat together with the heater electrode so as to lower humidity around the unit sensing unit.

### TECHNICAL FIELD

The present disclosure relates to a gas sensor that minimizes the effects of humidity.

### BACKGROUND ART

As the exposure to harmful air pollutants and environmental diseases become social issues, the government is making efforts to promote public health by improving the classification system of air pollutants that meets the purpose of air policy.

Recently, a temperature-humidity sensor, which is a kind of environmental sensor, has been adopted in smartphones. An interest in gas sensors is increasing particularly in smartphones or wearable devices.

The related art gas sensors are not suitable for use in smartphones and wearable devices, in view of sizes, power consumption, stability, sensitivity, response speed, etc. Therefore, the development of improved new gas sensors and packages is essential.

Accordingly, a Micro Electro-Mechanical Systems (MEMS) technology is used to manufacture gas sensors. In the gas sensor using the MEMS, wire bonding is performed to acquire an electrical signal from a sensor element. In order to perform the wire bonding, an electrode pad for the wire bonding must be provided on the sensor element and a separate electrode pad must be provided on a substrate on which the sensor element is attached. At this time, wire bonding is performed to connect an inner electrode pad and an electrode pad outside the package. Since wire bonding has a structure that is very vulnerable to vibration and external environment, various packaging methods such as flip chip bonding, BGA, or the like have recently emerged.

As the aforementioned packaging methods are developed, attempts to apply the gas sensor to various fields are continuing. However, since sensitivity of the gas sensor is very dependent to surrounding environments, it is difficult to apply the gas sensor to various fields. For example, since the sensitivity of the gas sensor depends on ambient humidity, it is difficult to use the gas sensor in an environment having a great humidity difference.

### DISCLOSURE

### Technical Problem

One aspect of the present disclosure is to provide a gas sensor capable of maintaining sensing sensitivity regardless of ambient humidity.

Another aspect of the present disclosure is to provide a gas sensor having gas selectivity.

### Technical Solution

In order to achieve the first aspect according to an implementation of the present disclosure may include a substrate, a unit sensing unit disposed on the substrate and including a wiring electrode, a heater electrode, and a gas sensing material, a protective cap provided with a side wall part formed on the substrate and surrounding the unit sensing unit, and a cover part disposed above the unit sensing unit and including at least one hole, and a heater unit disposed between the unit sensing unit and the cover part. The heater unit may generate heat together with the heater electrode so as to lower ambient humidity of the unit sensing unit.

In one embodiment, the heater unit may include a porous substrate, and a heater electrode deposited on the porous substrate.

In one embodiment, the side wall part may surround side surfaces of the porous substrate such that an external material is allowed to reach the unit sensing unit only through the porous substrate.

In one embodiment, a catalyst for promoting decomposition of a specific material may be applied to the porous substrate.

In one embodiment, the porous substrate may be provided with a plurality of layers.

In one embodiment, a heater electrode may be deposited on at least one of the plurality of layers.

In one embodiment, an average particle diameter of pores included in any one of the plurality of layers may be different from an average particle diameter of pores included in another layer different from the one layer.

In one embodiment, different types of catalysts may be applied to the plurality of layers, respectively.

### Advantageous Effects

According to the present disclosure, since a heater unit embedded in a gas sensor semi-permanently lowers ambient humidity, humidity effects with respect to the gas sensor can be minimized.

According to the present disclosure, since a type of gas that can reach a unit sensing unit can be limited according to a size of molecules, gas selectivity can be given to the gas sensor.

According to the present disclosure, since a type of gas that can reach the unit sensing unit can be limited to a gas that does not react with a specific catalyst, gas selectivity can be given to the gas sensor.

### BRIEF DESCRIPTION OF THE DRAWINGS

FIG. 1 is a cross-sectional view of a gas sensor according to the related art.

FIG. 2 is a cross-sectional view of a gas sensor according to the present disclosure.

FIG. 3A is a cross-sectional view of a unit sensing unit.

FIG. 3B is a planar view of the unit sensing unit.

FIGS. 4 and 5 are diagrams illustrating a modified implementation of a gas sensor according to the present disclosure.

FIG. 6 is a graph showing a detection signal of a gas sensor which does not include a heater unit.

FIG. 7 is a graph showing a detection signal of a gas sensor according to the present disclosure.

FIG. 8 is a graph showing sensitivity of a gas sensor according to concentration of ethanol gas.

FIG. 9 is a graph showing an improvement rate of sensitivity of a gas sensor according to concentration of ethanol gas.

### BEST MODE FOR CARRYING OUT PREFERRED EMBODIMENTS

Description will now be given in detail according to exemplary embodiments disclosed herein, with reference to the accompanying drawings. For the sake of brief description with reference to the drawings, the same or equivalent components may be provided with the same or similar reference numbers, and description thereof will not be repeated. In general, a suffix such as “module” and “unit” may be used to refer to elements or components. Use of such a suffix herein is merely intended to facilitate description of the specification, and the suffix itself is not intended to give any special meaning or function. In describing the present disclosure, if a detailed explanation for a related known function or construction is considered to unnecessarily divert the gist of the present disclosure, such explanation has been omitted but would be understood by those skilled in the art. The accompanying drawings are used to help easily understand the technical idea of the present disclosure and it should be understood that the idea of the present invention is not limited by the accompanying drawings.

It will be understood that when an element such as a layer, area or substrate is referred to as being “on” another element, it can be directly on the element, or one or more intervening elements may also be present.

The present disclosure relates to a gas sensor which minimizes effects or affection of humidity. Specifically, a gas sensor according to the present disclosure lowers humidity around the gas sensor in a heating manner, to maintain constant humidity around the gas sensor. Prior to describing the gas sensor according to the present disclosure, a method of reducing effects of humidity of the gas sensor according to the related art will be described.

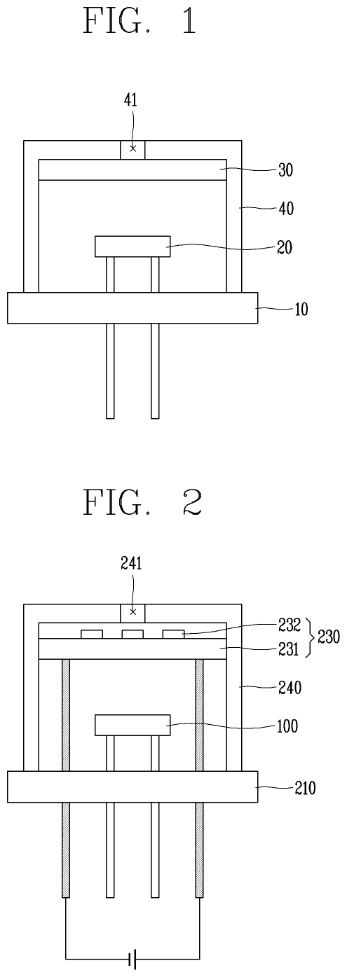

FIG. 1 is a cross-sectional view of a gas sensor according to the related art.

The related art gas sensor may include a substrate 10, a sensing unit 20, a moisture filter 30, and a protective cap 40.

Components constituting the gas sensor are disposed on the substrate 10. Wiring electrodes for connecting the gas sensor to an external power source and a controller may be provided on the substrate 10.

The sensing unit 20 generates a signal by reacting with specific gas. A gas sensing material included in the sensing unit 20 reacts with specific gas and thereby its resistance value changes. Accordingly, an amount of currents flowing through the sensing unit 20 may vary, and this change in the current amount becomes a gas detection or sensing signal.

The sensing unit 20 is covered with the protective cap 40. The protective cap 40 prevents foreign substances other than materials in a gaseous state from entering the sensing unit 20. External materials in a gaseous state may reach the sensing unit 20 through a hole 41 formed through the protective cap 40.

However, the gas sensing material provided in the sensing unit 20 may react with water vapor as well as the specific gas. For this reason, when ambient humidity of the gas sensor is high, sensitivity of the sensor may be lowered.

In order to prevent this, the related art gas sensor includes the moisture filter 30. The moisture filter 30 adsorbs water vapor around the gas sensor to prevent the water vapor around the gas sensor from reacting with the gas sensing material. However, an amount of water vapor that the moisture filter 30 can adsorb is limited. Due to this, when ambient humidity of the gas sensor is high, the moisture filter 30 is saturated and fails to exhibit its original function.

The present disclosure proposes a structure that can minimize effects of humidity of a gas sensor regardless of ambient humidity. Hereinafter, the present disclosure will be described in detail.

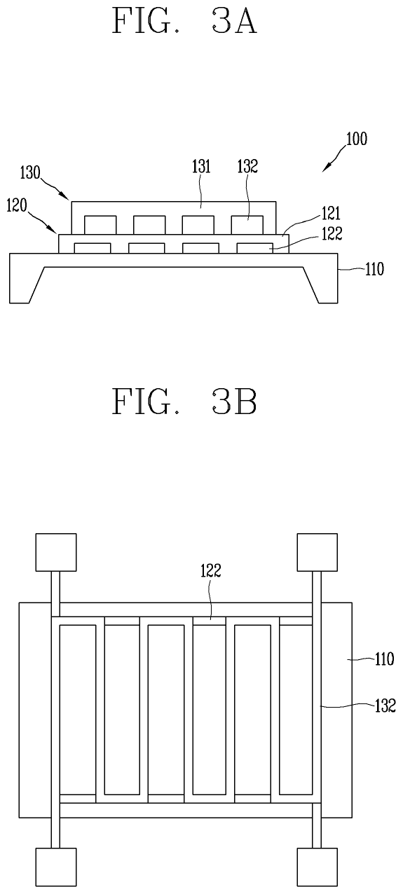

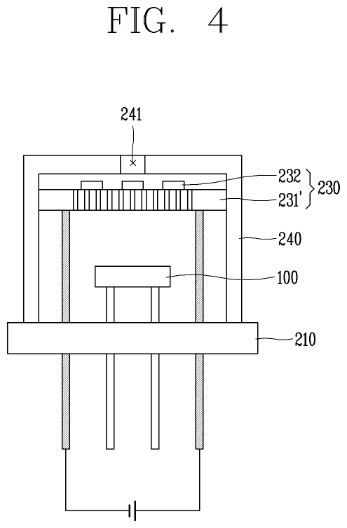

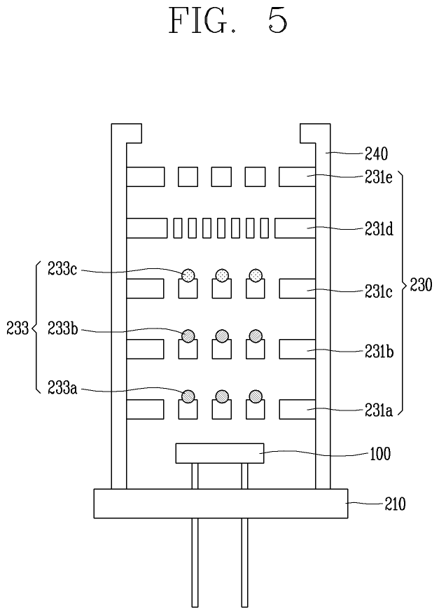

FIG. 2 is a cross-sectional view of a gas sensor according to the present disclosure, FIG. 3A is a cross-sectional view of a unit sensing unit, FIG. 3B is a planar view of the unit sensing unit, and FIGS. 4 and 5 are diagrams illustrating a modified implementation of a gas sensor according to the present disclosure.

A gas sensor according to the present disclosure may include a unit sensing unit 100, a substrate 210, a heater unit 230, and a protective cap 240.

A unit substrate 110 may be made of silicon. However, the present disclosure is not limited thereto, and any material capable of supporting components to be described later may be used as the unit substrate. All components of the unit sensing unit 100 are disposed on the unit substrate 110.

As illustrated in FIGS. 3A and 3B, a heater electrode 122 may be disposed on an upper surface of the substrate. However, the present disclosure is not limited thereto, and the heater electrode 122 may alternatively be disposed on a lower surface of the substrate. This will be described later.

The heater electrode 122 may be made of any one of platinum and tungsten. However, the present disclosure is not limited thereto, and any material that generates heat when a voltage is applied may be used as the heater electrode.

The heater electrode 122 serves to supply thermal energy to a sensing material to be described later. The sensor according to the present disclosure can control an amount of thermal energy supplied to the sensing material by adjusting a voltage applied to the heater electrode 122, and thus, temperature of the sensing material can be controlled.

Meanwhile, an insulating layer 121 insulates the heater electrode 122 from other electrodes. The insulating layer 121 may be made of silicon oxide. However, the present disclosure is not limited thereto, and any material capable of insulating the heater electrode 122 from other electrodes may be used.

A sensing electrode 132 may be disposed on the insulating layer 121. In this case, the insulating layer 121 insulates the heater electrode 122 and the sensing electrode 132 from each other.

Although not shown, the heater electrode 122 may be disposed on the lower surface of the unit substrate 110. In this case, the sensing electrode 132 may be disposed on the upper surface of the unit substrate 110. In this structure, the heater electrode 121 and the sensing electrode 131 are disposed with the unit substrate 110 interposed therebetween.

The sensing electrode 132 outputs a signal generated due to changes in electrical properties of a gas sensing material 131. Here, the output signal may be a resistance change, a current change, or the like. A signal output from a sensor array according to one implementation of the present disclosure means the signal output from the sensing electrode 132.

Meanwhile, the gas sensing material 131 is disposed to cover the sensing electrode 132. The gas sensing material 131 is made of tin oxide as a main component, and may be made by mixing a metal such as platinum, lead, or nickel, an oxide of the metal, polymers, and organic compounds.

When thermal energy is applied to the gas sensing material 131 from the heater electrode 122, free electrons of the gas sensing material 131 increase, and oxygen in the atmosphere is adsorbed on the gas sensing material 131 by the increased free electrons. Accordingly, potential barriers are formed on tin oxide particles forming the gas sensing material 131, thereby lowering electrical conductivity between the particles.

In this state, when the oxygen adsorbed on the gas sensing material 131 reacts with specific gas, the electrical conductivity of the gas sensing material 131 increases again. The sensing electrode 132 outputs a signal generated as the oxygen adsorbed on the gas sensing material 131 is desorbed.

As described above, sensitivity of the sensor increases as a difference between an adsorption amount and a desorption amount of oxygen increases. For this purpose, temperature of the tin oxide must be raised. A temperature at which the sensitivity of the sensor is maximized depends on a type of gas to be sensed.

The unit sensing unit 100 is disposed on the substrate 210. In addition, components constituting the gas sensor according to the present disclosure may be disposed on the substrate 210.

The unit sensing unit 100 is covered with the protective cap 240. The protective cap 240 prevents external materials other than materials in a gaseous state from entering the unit sensing unit 100. Specifically, the protective cap 240 includes a side wall part formed on the substrate 210 to surround the unit sensing unit, and a cover part disposed on a top of the unit sensing unit 100 and having at least one hole 241. An external material in a gaseous state may reach the unit sensing unit 100 through the hole 241.

On the other hand, the heater unit 230 is disposed between the unit sensing unit 100 and the cover part. The heater unit 230 generates heat together with the heater electrode 122 to lower humidity around the unit sensing unit 100.

The heater unit 230 is located above the unit sensing unit 100 to increase temperature so as to prevent external vapor from flowing into the sensor. Since the heater unit 230 lowers ambient humidity of the gas sensor without adsorbing vapor, the heater unit 230 can semi-permanently reduce the effects or influence of the humidity to the gas sensor.

Meanwhile, the heater unit 230 may include a support substrate 231 and a heater electrode 232 deposited on the support substrate 231. At least one hole may be formed through the support substrate 231. External gas introduced into the gas sensor through the hole 241 of the protective cap 240 may reach the unit sensing unit 100 through the hole formed through the support substrate 231.

Meanwhile, as illustrated in FIG. 4, the support substrate 231 may be implemented as a porous substrate 231′. Since the porous substrate 231′ includes a plurality of pores, an external material in a gaseous state can pass through the porous substrate 231′. In one implementation, the porous substrate 231′ may be a porous alumina substrate.

On the other hand, an average particle diameter of the pores included in the porous substrate 231′ can be adjusted when manufacturing the porous substrate 231′. A type of gas that can pass through the porous substrate 231′ may differ depending on a size of the pores included in the porous substrate 231′. By using this, gas selectivity can be given to the gas sensor.

Specifically, the side wall part provided on the protective cap 240 may surround side surfaces of the porous substrate so that an external material can reach the unit sensing unit 100 only through the porous substrate 231′. A material that can reach the unit sensing unit 100 should have a size smaller than the size of the pores included in the porous substrate. The porous substrate 231′ blocks molecules larger than the pores included in the porous substrate. With this configuration, the present disclosure can give gas selectivity to the gas sensor.

Meanwhile, the present disclosure may provide gas selectivity to the gas sensor by utilizing the porous substrate and a catalyst. Specifically, referring to FIG. 5, a catalyst 233 may be applied or coated on the porous substrate to promote decomposition of a specific material. When the catalyst 233 is applied to the porous substrate 230, a contact area between the catalyst 233 and an external material may increase, and the performance of the catalyst 233 may be improved. As a result, gas decomposed by the catalyst 233 may not reach the unit sensing unit 100. With this configuration, the present disclosure can give gas selectivity to the gas sensor.

Meanwhile, the present disclosure may provide gas selectivity to the gas sensor by utilizing a plurality of porous substrates. Specifically, the porous substrate may be provided with a plurality of layers.

A heater electrode may be deposited on at least one of the plurality of layers. When the heater electrode is deposited on the plurality of layers, the effects of humidity with respect to the gas sensor can be greatly reduced.

Meanwhile, referring to FIG. 5, sizes of pores included in each of the plurality of layers 231a to 231e may be different from one another. Specifically, an average particle diameter of the pores included in any one of the plurality of layers 231a to 231e may be different from an average particle diameter of the pores included in another layer different from the one layer.

When a layer closer to the unit sensing unit 100 has a smaller average particle size, only a very limited type of gas can reach the unit sensing unit 100. With this configuration, the present disclosure can give gas selectivity to the gas sensor.

Meanwhile, different types of catalysts 233a to 233c may be applied to the plurality of layers, respectively. In this case, since only a gas which is not decomposed by the catalysts applied to the respective plurality of layers can reach the unit sensing unit 100, gas selectivity of the gas sensor can be enhanced.

Hereinafter, effects of humidity with respect to the gas sensor according to the present disclosure will be described.

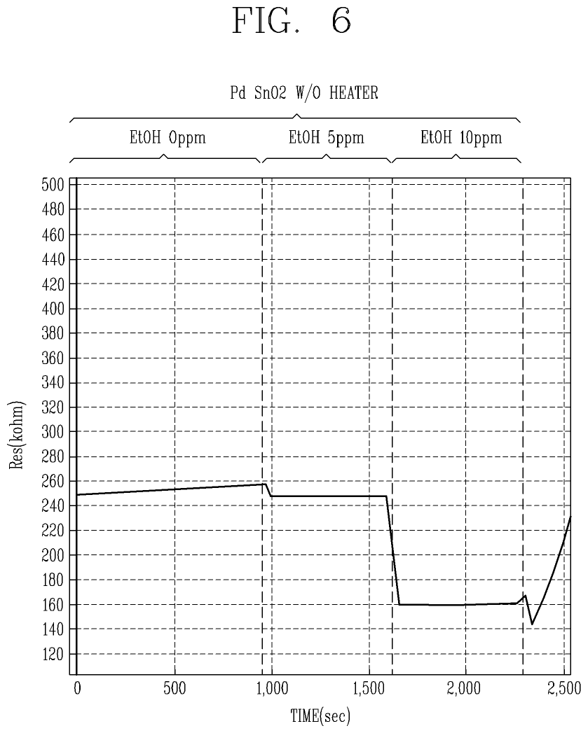

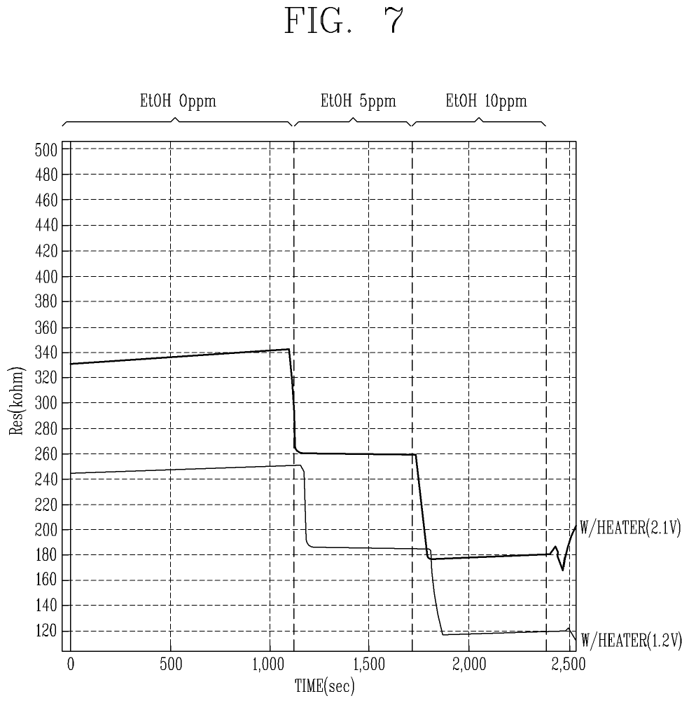

FIG. 6 is a graph showing a sensing or detection signal of a gas sensor which does not include a heater unit, and FIG. 7 is a graph showing a sensing or detection signal of a gas sensor according to the present disclosure.

In order to measure the effects of humidity with respect to the gas sensor, a signal generated from the gas sensor was measured while changing concentration of ethanol gas at a condition of humidity of 70%. Three types of sensors were used in this experiment. Specifically, the three types of sensors are a sensor without a heater unit (hereinafter, referred to as Comparative example), a sensor applying a voltage of 1.2 V to a heater unit (hereinafter, referred to as Example 1), a sensor applying a voltage of 2.1 V to a heater unit (hereinafter, referred to as Example 2)). Here, in all of Comparative Example, and Examples 1 and 2, both Pd and SnO2 are included as gas sensing materials.

Referring to FIG. 6, a signal generated when the concentration of ethanol gas is 5 ppm and a signal generated when the concentration of ethanol gas is 10 ppm are clearly distinguished from each other. However, a signal generated when the concentration of ethanol gas is 0 ppm and the signal generated when the concentration of ethanol gas is 5 ppm are not distinguished well from each other. In this manner, it can be confirmed in Comparative example that sensitivity is not good under such high humidity condition.

On the other hand, referring to FIG. 7, a signal generated when the concentration of ethanol gas is 5 ppm and a signal generated when the concentration of ethanol gas is 10 ppm are clearly distinguished from each other, and also a signal generated when the concentration of ethanol gas is 0 ppm and the signal generated when the concentration of ethanol gas is 5 ppm are clearly distinguished from each other. In this manner, it can be confirmed that the gas sensor according to the present disclosure has high sensitivity even under the high humidity condition.

On the other hand, it can be seen that a reaction rate with respect to an external material becomes faster when a higher voltage is applied to the heater unit (compare Examples 1 and 2).

On the other hand, sensitivity and sensitivity improvement rate in Comparative Example, and Examples 1 and 2 were measured while gradually increasing the concentration of ethanol gas.

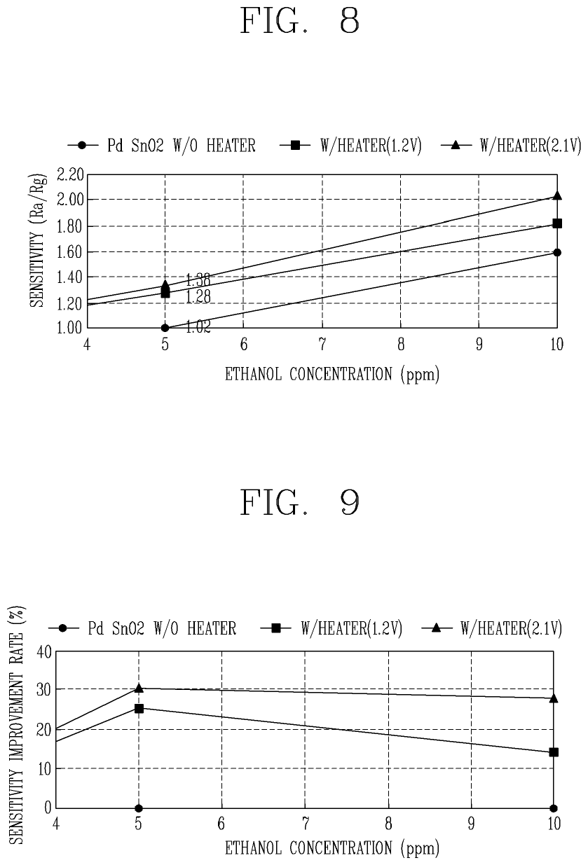

FIG. 8 is a graph showing sensitivity of the gas sensor according to the concentration of ethanol gas, and FIG. 9 is a graph showing an improvement rate of sensitivity of the gas sensor according to the concentration of ethanol gas.

Referring to FIG. 8, it can be seen that the sensitivity is the highest in Example 2 regardless of the concentration of ethanol gas, and ethanol of 5 ppm or less is not sensed in Comparative example.

On the other hand, referring to FIG. 9, it can be seen in Examples 1 and 2 that the sensitivity is the maximum when the concentration of ethanol gas is 5 ppm, and it can also be seen in Comparative example that the sensitivity is constant regardless of the concentration of ethanol.

It will be apparent to those skilled in the art that the present disclosure may be embodied in other specific forms without departing from the essential characteristics thereof.

Therefore, it should also be understood that the above-described embodiments are not limited by any of the details of the foregoing description, unless otherwise specified, but rather should be construed broadly within its scope as defined in the appended claims, Therefore, all changes and modifications that fall within the metes and bounds of the claims, or equivalents of such metes and bounds are therefore intended to be embraced by the appended claims.

1. A gas sensor comprising:
a substrate;
a unit sensing unit disposed on the substrate and provided with a wiring electrode, a heater electrode, and a gas sensing material;
a protective cap provided with a side wall part formed on the substrate and surrounding the unit sensing unit, and a cover part disposed above the unit sensing unit and having at least one hole; and
a heater unit disposed between the unit sensing unit and the cover part,
wherein the heater unit generates heat together with the heater electrode to lower ambient humidity of the unit sensing unit.

2. The gas sensor of claim 1, wherein the heater unit comprises:
a porous substrate; and
a heater electrode deposited on the porous substrate.

3. The gas sensor of claim 2, wherein the side wall part surrounds side surfaces of the porous substrate such that an external material is allowed to reach the unit sensing unit only through the porous substrate.

4. The gas sensor of claim 3, wherein a catalyst for promoting decomposition of a specific material is applied to the porous substrate.

5. The gas sensor of claim 3, wherein the porous substrate is provided with a plurality of layers.

6. The gas sensor of claim 5, wherein a heater electrode is deposited on at least one of the plurality of layers.

7. The gas sensor of claim 5, wherein an average particle diameter of pores included in any one of the plurality of layers is different from an average particle diameter of pores included in another layer different from the one layer.

8. The gas sensor of claim 5, wherein different types of catalysts are applied to the plurality of layers, respectively.
